(19) United States
(12) Patent Application Publication
(10) Pub. No.: US 2007/0178525 A1
(43) Pub. Date: Aug. 2, 2007

(54) Livestock health management

(75) Inventors: William E. Hessman, Sublette, KS (US); Timothy Murphy, Dodge City, KS (US); Roy N. Brown, Satanta, KS (US); Ronald C. Shortridge, Garden City, KS (US)

Correspondence Address: MIRICK, O'CONNELL, DEMALLIE & LOUGEE, LLP, 1700 WEST PARK DRIVE, WESTBOROUGH, MA, 01581, US

(73) Assignee: Central States Testing, LLC, Satanta, KS (US)

(21) Appl. No.: 11/340,958

(22) Filed: Jan. 27, 2006

(51) Int. Cl. C12Q 1/70; G01N 33/53

(52) U.S. Cl. 435/7.1; 435/5

(57) ABSTRACT

The present invention provides methods for managing livestock to improve performance and health of the general livestock population by reducing the impact of subclinical animals persistently infected with a contagious disease by separating animals into arrival groups, screening all animals for the pathogen of the contagious disease, promptly removing the pathogen positive animals from the general livestock population and feeding and managing the pathogen negative animals and pathogen positive animals separately. In preferred embodiments, the pathogen positive animals are removed from their arrival group within about 1.5-3 days after arrival. In preferred embodiments, the method of the present invention is applied in a cattle operation such as a seedstock operation, a cow-calf operation, a stocker operation, a backgrounding operation, a feedlot operation, a dairy operation, a farm of origin, such as a dairy farm, an auction facility, a gathering point or a buyer facility.

### FIELD OF THE INVENTION

The present invention relates to methods for managing livestock to improve the performance and health of the general livestock population by reducing the impact of clinically inapparent animals that are persistently infected with a contagious disease. More specifically, the invention relates to reducing the impact of cattle that are persistently infected with bovine viral diarrhea virus in a cattle operation such as a seedstock operation, a cow-calf operation, a stocker operation, a backgrounding operation, a feedlot operation, a dairy operation, a farm of origin, such as a dairy farm, an auction facility, a gathering point or a buyer facility.

### BACKGROUND OF THE INVENTION

Bovine viral diarrhea virus (BVDV) is a positive stranded RNA virus of the Pestivirus genus of the Flaviviridae family. This virus is found worldwide in ruminant animals both domestic and wild, including sheep, goats and deer as well as cattle. In addition to having several genetic subtypes (BVDV 1a, BVDV 1b and BVDV 2), the virus can exist in cytopathic and noncytopathic forms, producing a wide range of clinical disease. The interactions of this complex pathogen with the immune system at different stages of life are responsible for production of animals persistently infected (PI) with BVDV as well as a wide range of clinical diseases ranging from subclinical or clinically inapparent infections to a highly fatal disease known as mucosal disease (Baker, J. C., The clinical manifestations of bovine viral diarrhea infection, Vet Clin North Am Food Anim Pract, 111(3): 425-445, 1995). The initial description of the disease and pathogen were based on an enteric infection on a dairy farm, resulting in the names bovine viral diarrhea (BVD) and bovine viral diarrhea virus. It is now recognized that BVDV is responsible for a number of disease syndromes in cattle affecting the reproductive, respiratory tract, gastrointestinal, circulatory, immunologic, lymphatic, musculoskeletal, integumentary and the central nervous systems.

It is believed that the continuum of these disease syndromes comes from the production of cattle immunotolerant to BVDV. These animals are persistently infected (PI) with BVDV and are lifelong carriers. PI animals are produced when a dam is exposed to the virus when she is approximately 40-125 days into gestation. The virus crosses transplacentally to the fetus. It is during this time frame in which the fetus is becoming immunocompetent by recognizing what is “self” so at later times the immune system can mount an immune response against anything not recognized as “self”. When BVDV is present at this time of gestation, the developing immune system of the calf recognizes BVDV as “self” and therefore does not mount an immune response against the virus. As a result, the calf continues to produce and shed BVDV for the remainder of its life, potentially exposing all cattle it contacts.

Persistently infected animals are persistently viremic animals. Cattle persistently infected with BVDV act as the major reservoir of BVDV and are the primary source of infection, with transiently infected cattle considered a less important source. See generally, Larson, R. L., et al., Bovine Viral Diarrhea (BVD): Review for Beef Cattle Veterinarians, Bov Pract 38:93-102, 2004. Persistently infected animals are much more efficient transmitters of BVDV than transiently infected animals because they secrete much higher levels of virus for a much longer period of time.

After a short incubation period, transiently infected animals become viremic and virus may be shed in body secretions and excretions from days 4 to 15 post-infection FIG. 1 is a schematic diagram providing a generalized illustration of BVDV effects on immune function resulting in leukopenia. The white blood cell count is plotted against days post-infection. In the first week post-infection there is an absence of clinical signs and symptoms. Acute BVD is often (70-90% of the time) subclinical. BVD associated disease following the acute BVD may be clinical or subclinical. The details of the interactions of the BVD virus with both innate and adaptive immunity are reviewed in Chase, C. C. L., et al., The immune response to bovine viral diarrhea virus: a constantly changing picture, Vet Clin North Am Food Anim Pract 20:95-114, 2004.

In contrast, PI animals usually have a very high and persistent viremia. BVDV is shed throughout life from virtually all secretions and excretions including nasal discharge, saliva, semen, urine, tears, milk, and to a lesser extent, feces. Traven, M., et al., Primary bovine viral diarrhoea virus infection in calves following direct contact with a persistently viraemic calf. J Vet Med B 38:453-462, 1991. Fetuses, placentae and fetal fluids, from BVDV-induced abortions can also contain BVDV. Horizontal transmission of BVDV to seronegative cattle has been shown to occur after only one hour of direct contact with a single PI animal. Over-the-fence contact with a PI animal from a neighboring herd can also introduce BVDV into a susceptible herd (Larson et al., 2004).

Traditional control and prevention of BVDV infections in a herd relies on biosecurity, identification and removal of PI cattle and vaccination (Baker, J. C., Control and prevention of BVDV infections, Michigan Dairy Review, 1(4): 16, 1996). Identification of an animal testing positive for BVDV virus as persistently infected requires a second test four to six weeks later, since a single positive test cannot discriminate between acute infection and persistent infection (Baker, 1996). Herd screening for persistent infection with BVDV may be most applicable for herds that have a current and confirmed BVDV problem, but several factors must be considered before undertaking screening in herds without a BVDV problem (Baker, 1996). Larson et al. have stated that screening cattle for the presence of PI individuals prior to purchase or at arrival [in stocker/feedlot operations] has not been adequately evaluated for economic return (Larson, R. L., et al., Bov Pract 38:93-102, 2004).

### SUMMARY OF THE INVENTION

The present invention provides methods for managing livestock to improve the performance and health of the general livestock population by reducing the impact of clinically inapparent animals that are persistently infected with a contagious disease. The benefits of the method of the present invention include reduced mortality and improved feeding performance in the animals that initially are uninfected and are isolated from contact from animals persistently infected with a contagious disease. In preferred embodiments, the method of the present invention is applied to livestock in a cattle operation such as a seedstock operation, a cow-calf operation, a stocker operation, a backgrounding operation, a feedlot operation, a dairy operation, a farm of origin, such as a dairy farm, an auction facility, a gathering point or a buyer facility. The cattle operation may be a beef cattle operation or a dairy cattle operation.

In preferred embodiments of the method of the present invention, the livestock, such as cattle, are kept separated by source in arrival groups. All livestock, including all apparently healthy animals, are prophylacticly screened to provide prognostic information regarding the health and feedlot performance of the arrival groups. In preferred embodiments, the prophylactic screening is performed using a method that reduces the number of tests (and thus the costs) required to screen the entire population consistent with the benefits obtained. In preferred embodiments, test results are reported about twelve to about twenty-four hours after the testing laboratory receives the samples to be screened. The animals that test positive for the pathogen are promptly removed from contact with the general livestock population, preferably removed from their arrival group-in about 1.5 to about 3 days after arrival at the cattle operation. In preferred embodiments, such as a feedlot operation, both the removed pathogen positive animals and the pathogen negative animals are fed and managed to the desired endpoints.

The method of the present invention involves the steps of separating the animals into arrival groups, providing a unique identifier for each animal in a livestock population; testing each animal to detect pathogen positive animals; promptly removing the detected pathogen positive animals from their arrival group. In certain preferred embodiments, the step of testing includes the steps of collecting a sample; coding the sample to correspond to the animal identifier; mixing aliquots of the coded samples of a group of animals to form a pooled sample; testing the pooled sample for evidence of a persistently infected animal; and further testing the individual samples that contributed to a pooled sample that showed evidence of at least one pathogen positive animal in the group of animals corresponding to that pooled sample. In certain preferred embodiments, the method also includes the steps of vaccinations, deworming, castration, administering growth promotants, giving prophylactic antimicrobials, tipping horns, adjust cattle to high energy diets and alleviating any sickness that comes with commingling, transport and the other stresses of procurement.

In other preferred embodiments, the present invention provides a method of rapid testing for pathogen positive individual animals within a population of livestock comprising the steps of combining aliquots of samples from individual animals to form pooled samples; testing the pooled samples to produce a set of first results, thereby identifying a subset of putative positive samples; testing aliquots from the putative positive samples to produce a set of second results, identifying the individual animals that had been combined to selecting the pooled samples producing positive first results thereby identifying subclinical pathogen positive individual animals and individual animals negative for the pathogen. In preferred embodiments, the time elapsed between receiving the samples and reporting the test results is about twelve to about twenty-four hours, preferably about twelve hours.

### BRIEF DESCRIPTION OF THE DRAWINGS

FIG. 1 is a schematic diagram providing a generalized illustration of BVDV effects on immune function resulting in leukopenia. The white blood cell count is plotted against days post-infection. In the first week post-infection there is an absence of clinical signs and symptoms. Acute BVD is often (70-90% of the time) subclinical. BVD associated disease following the acute BVD may be clinical or subclinical.

FIG. 2 is a schematic diagram illustrating the classification scheme underlying the designation of the PI, PIR, NPIE, NPIER and NPIU groups by position of the pens within the feedlot. In alley 1, the PI animals identified by the prophylactic screening as pathogen positive were left in the pen with their arrival group, and such a pen is indicated as a PI pen in FIG. 2. In general, PI pens were flanked by pens that did not include PI animals and since they were adjacent to a PI pen, they were designated as NPIE pens. NPIU pens were the remaining pens that neither contained a PI animal on arrival nor were adjacent to a PI pen.

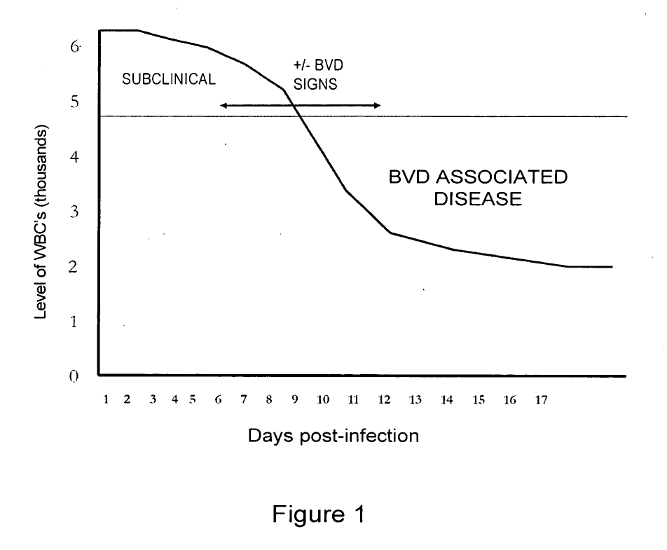

In contrast, the PI animals in the pens of alleys 2 and 3 were removed to quarantine pens at the end of alley 3 after screening of all animals. The pens that had contained a PI animal that had been removed to quarantine were designated as PIR pens, and flanking pens that had not contained a PI animal were designated NPIER pens. The pens in alleys 2 and 3 that had neither contained a PI animal nor were adjacent to a pen that had contained a PI animal were designated NPIU pens. Each alley had a hospital that was used to treat animals from pens in that alley.

All of the pens were filled and emptied at least twice during the course of the study, and thus the diagram illustrates the classification scheme, and does not necessarily show a snapshot of the distribution of animals within the feedlot at any given time. Some pens changed their status from NPIU to NPIE or NPIU to NPIER over the course of the study if the status of an adjacent pen changed to PI or PIR, respectively.

FIG. 3A is a graphic representation comparing the average PI prevalence and average weight-in over the course of a year. FIG. 3B is a graphic representation comparing the average PI prevalence and mortality over the course of a year. A significant correlation between PI prevalence and either average weight-in or mortality was not observed.

FIG. 4 is a schematic diagram of the first days after the arrival of the livestock at the cattle operation illustrating the timing of ear notch sampling, reporting of the BVDV antigen capture ELISA test results and removal of the individual BVDV positive individual animals.

FIG. 5 is a schematic diagram of a preferred embodiment of the livestock health management method of the present invention, showing the steps of keeping the livestock separated by source on arrival in the feedlot 100; performing intake processing, including ear notch sampling 200; performing prophylactic screening for BVDV infection 300; removing BVDV positive animals to quarantine 400; and feeding and managing BVDV negative animals 500 and BVDV positive animals 520 separately. In certain preferred embodiments, the method also includes the step of selling the animals 600, 620.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

As used herein, “average daily gain” (ADG) means the total number of pounds gained during the feeding period divided by days on feed (DOF), the number of days an animal spent in the feedlot.

As used herein, “cost of gain” (COG) means the total feedlot-related costs (feed, yardage, processing, medicine, death loss) divided by total gain during the feeding period. COG can be calculated on a ‘deads-in’ or ‘deads-out’ basis.

As used herein, “feed conversion” (F/G) means the amount of feed consumed by the animal divided by the number of pounds gained. Feed conversion is conveniently calculated on a dry matter basis after allowance for the moisture content of the feed.

As used herein, “railers” are cattle that are sold due to illness or injury prior to being finished.

As used herein “cattle operation” includes dairy cattle operations as well as beef cattle operations, such as a seedstock operation, a cow-calf operation, a stocker operation, a backgrounding operation, a feedlot operation, a dairy operation, a farm of origin, such as a dairy farm, an auction facility, a gathering point or a buyer facility.

As used herein, “subclinical” means without clinical manifestations; said of the early stage(s) of an infection or other disease or abnormality before symptoms and signs become apparent or detectable by clinical examination or laboratory tests, or of a very mild form of an infection or other disease or abnormality. As used herein, “subclinical” encompasses PI animals that do not show clinical signs and symptoms, but having a viral load that can be detected using a suitably sensitive laboratory test. With reference to the BVDV, a PI animal by definition cannot respond immunologically to the particular BVDV strain it is persistently infected with, although they can respond to heterologous strains. However, when a PI animal that is persistently infected with a non-cytopathic (NCP) BVDV strain becomes super-infected with an antigenically homologous cytopathic (CP) BVDV strain, the clinical condition, mucosal disease (MD) can develop. “Subclinical” as used herein is equivalent to “clinically inapparent” (Drew, T., “Bovine Viral Diarrhoea,” Chapter 2.10.6 in Manual of Diagnostic Tests and Vaccines for Terrestrial Animals, 5^{th} Ed., 2004, World Organisation For Animal Health (Office International des Epizooties, OIE), http://www.oie.int/eng/normes/mmanual/A_{—}00132.htm).

As used herein PI is used to refer to animals that have tested positive for the pathogen upon a single screening test of a sample from that animal, even though proper veterinary practice may require a subsequent sampling and testing four to six weeks later to diagnose a PI individual. While a small fraction of pathogen positive individual animals may be acutely infected, and thus a less-serious source of infection than PI animals, it is advantageous in the practice of the method of the present invention to treat all pathogen positive animals as if they were persistently infected.

### EXAMPLE 1

### Effects of Exposure to PI Calves on Weight Gain of Non-PI Calves

This study evaluated the effects of keeping calves separated by source and shipment to limit the exposure of non-PI calves to PI calves. The study enrolled 2,284 head of “high-risk” calves. In general, “high-risk” is used to refer to cattle that are expected to experience significant morbidity (≧20%) and/or mortality (1-1.5% death loss) when received at the feedlot.

The 5 cwt. calves (weighing about 500 pounds each) came from the southeastern United States through five different order buyers. Upon arrival, the calves were kept separated according to load and/or buyer, placed into lots and distributed in 24 study pens that contained 60-150 head each. All calves were ear-notched at initial processing and PI tested using the immunohistochemistry (IHC) test. The study was carried out over nine months from October 2003 to June 2004.

Seven PI calves were found in the study group, indicating a prevalence of 0.31% or 3.1/1,000. This prevalence is consistent with a estimated prevalence of 0.3% reported in a recent study of 2,000 cattle on arrival at feedlots (Loneragan, G. H., et al., Prevalence, outcome, and health consequences associated with persistent infection with bovine viral diarrhea virus in feedlot cattle, J Am Vet Med Assoc. 226(4):595-601, 2005). The seven PI calves were found in five of the 24 test pens. Three pens had one PI calf each and two pens had two PI calves each. Five of the seven PI animals (71%) survived to slaughter, and three of the seven PI animals (43%) required antibiotic therapy. The PI and non-PI pens were randomly placed throughout the feed yard, with the PI status of adjacent pens unknown. Health and performance parameters were tracked through the finishing period (Tables 1 and 2, below). Feed yard workers were not told if the calves in a given pen were PI positive or PI negative.

There was no significant difference (p=0.76) in the starting average weight of PI calves (574 pounds) compared to non-PI calves (570 pounds). However, the study results showed differences that were significant at the p≦0.05 level between calves raised in the PI pens and those raised in the Non-PI pens in the feedlot performance parameters of weight gain (357 v. 405 lbs), feed conversion (F/G) and cost of gain (COG). A difference in average daily gain (ADG) was observed that was close to, but not at the criterion level for significance (p=0.07).

TABLE 1
Performance of calves in PI pens and non-PI pens

| | | # | Wt | Wt Out | Wt Out | Wt | | | Consump | F/G | COG |
|---|---|---|---|---|---|---|---|---|---|---|---|
| | Head | Pens | In | Deads Out | Deads In | Gain | DOF | ADG | Dry | Deads In | Deads In |
| NPI | 1731 | 19 | 571 | 1051 | 976 | 405 | 180 | 2.25 | 14.02 | 6.26 | .691 |
| PI | 553 | 5 | 574 | 1040 | 931 | 357 | 177 | 2.00 | 13.75 | 6.94 | .767 |

| | | | | | | | |
|---|---|---|---|---|---|---|---|
| P-value | .70 | .04 | .58 | .07 | .53 | .02 | .05 |

The average difference in the COG between PI and non-PI pens was $7.60/cwt. gained. Therefore, cattle that did not have a PI animal in their pen had a $30.78/head cost advantage (405 lbs. gained, $0.076/lb. advantage) over cattle with a PI animal in their pen. The effect of a non-PI pen being adjacent to a PI pen was not investigated. Since an effect of the spatial relation of a PI pen adjacent to control group (NPI pen) was not known, this cost advantage per head would most likely be a conservative figure.

TABLE 2
Health of calves in PI pens and non-PI pens

| | | | First | Second | Average No. | | Med | | |
|---|---|---|---|---|---|---|---|---|---|
| | Head | % | relapse | relapse | of | Med | $/Head | % | % |
| | count | Morbidity | rate | rate | treatments | $/Head | treated | Mortality | Railers |
| NPI Pens | 1,731 | 49.52 | 46.04 | 55.44 | 1.72 | 25.40 | 52.80 | 6.96 | 6.34 |
| PI Pens | 553 | 42.31 | 43.74 | 54.84 | 1.68 | 23.05 | 55.41 | 10.37 | 6.39 |
| p-value | | 0.22 | 0.71 | 0.93 | 0.76 | 0.43 | 0.24 | 0.14 | 0.97 |

Table 3 compares the cumulative mortality of PI and non-PI calves on a monthly basis over the first five months in the feedlot and at closeout. In the first month (<31 days on feed, DOF), the mortality rate of the PI calves was more than twice that of the non-PI calves, a difference that was significant at the p≦0.05 level. However, the difference became smaller and did not reach the criterion level of significance as the study continued.

TABLE 3
Mortality by DOF

| | <31 DOF | | <61 DOF | | <91 DOF | | <121 DOF | | <151 DOF | | Close Out | |
|---|---|---|---|---|---|---|---|---|---|---|---|---|
| | % | % DIFF | % | % DIFF | % | % DIFF | % | % DIFF | % | % DIFF | % | % DIFF |
| PI | 4.95 | +144 | 0.23 | +83 | 9.91 | +69 | 10.0 | +56 | 10.1 | +51 | 10.3 | +49 |
| NPI | 2.03 | | 5.03 | | 5.88 | | 6.43 | | 6.68 | | 6.90 | |
| P VALUE | .04 | | .11 | | .18 | | .26 | | .28 | | .30 | |

Table 4 compares the mortality diagnosis of PI and non-PI calves on a monthly basis over the first five months in the feedlot and at closeout. Bovine respiratory disease (BRD) was the most common cause of death in both groups, and was significantly more common in the PI groups during the first four months.

TABLE 4
Mortality Diagnosis by DOF

| | DIAGNOSIS | <31 DOF | <61 DOF | <91 DOF | <121 DOF | <151 DOF | Close Out |
|---|---|---|---|---|---|---|---|
| PI | BRD | 4.42% | 7.94% | 8.62% | 8.73% | 8.73% | 8.96% |
| | HISTOPHILOSIS | 0.0% | .34% | .34% | .34% | .34% | .34% |
| | METABOLIC | 0.2% | 0.2% | 0.2% | 0.2% | 0.4% | 0.4% |
| | OTHER | 0.5% | 0.5% | 0.5% | 0.5% | 0.5% | 0.5% |
| | BVD | 0 0% | 0 2% | 0.2% | 0.2% | 0.2% | 0.2% |
| NPI | BRD | 1.5% | 4.1% | 4.5% | 49% | 49% | 5.1% |
| | | (P = .003) | (P = .01) | (P = .03) | (P = .05) | (P = .06) | (P = .07) |
| | HISTOPHILOSIS | 0.1% | 0.2% | .02% | 0.2% | 0.2% | 0.2% |
| | METABOLIC | 0.1% | 0.3% | 0.4% | 0.5% | 0.6% | 0.7% |
| | OTHER | 0.4% | 0.5% | 0.5% | 0.6% | 0.6% | 0.7% |
| | BVD | 0.1% | 0.1% | 0.1% | 0.1% | 0.1% | 0.1% |

In summary, the results showed that a PI animal causes a distinct increase in mortality in the PI pen, with the majority of the mortality increase occurring in the first 30 days on feed. However, the difference in the percent mortality between PI and NPI pens in later periods was not significant at the p≦0.05 level. Thus, in this study, the increased costs associated with exposure of a NPI calf to a PI calf are primarily due to poorer feed conversion. The results showed that animals in pens with PI animals converted 11% less efficiently than NPI pens and the COG in PI pens was 11% greater. Other studies have shown an increased morbidity associated with PI animals, but the present results showed an increase in morbidity in the NPI pens that was not significant at the p≦0.05 level. Similarly, the average number of treatments was higher in the NPI pens than in the PI pens, but the difference was not statistically significant at the p≦0.05 level.

### EXAMPLE 2

### Effects of Prompt Quarantine of PI Calves on Weight Gain of Non-PI Calves

The study enrolled nearly 22,000 head of “high-risk” 5 cwt calves that came to a commercial starter yard (Cattle Empire LLC, Satanta, Kans.) from ten states south and east of Kansas. As in the previous study described above in Example 1, the calves were kept separated on arrival according to load and/or buyer, placed into lots and distributed in study pens that contained 60-150 head each. All animals were subjected to prophylactic screening within a few days of arrival, and the pens were characterized by the presence or absence of PI (i.e., BVDV positive) animals on arrival and whether those BVDV positive animals were left in the pen or removed to quarantine pens when the BVDV status was determined by the prophylactic screening. Typically, BVDV positive animals were removed within one day after the screening results were available, and about 48-72 hours after sampling.

All calves remained with their arrival groups. Each pen of calves was classified in one of five treatment groups as defined in Table 5, below, based on the results of the prophylactic screening of the calves in that pen.

TABLE 5
Study Treatment Groups

| | |
|---|---|
| PI Group | at least one BVDV positive animal in the pen, and that |
| | animal was left in that pen. |
| PIR Group | at least one BVDV positive animal in the pen, and that |
| | animal was removed from the pen within 48 hours of |
| | testing. |
| NPIE Group | Non-PI pen but exposed. No BVDV positive animal in |
| | the group on arrival, but this pen was placed next to a |
| | PI pen. Therefore the Non-PI group had direct exposure |
| | to a BVDV positive animal in the adjacent pen through |
| | the fence. |
| NPIER Group | Non-PI pen but animals had possible exposure to a |
| | BVDV positive animal. No BVDV positive animal on |
| | arrival but possible exposure to BVDV from being placed |
| | next to a PIR group. The exposure would come from any |
| | acute BVDV infections in the PIR group originating |
| | from the BVDV positive animal spreading BVDV to its |
| | penmates prior to its removal. |
| NPIU Group | Non-PI pen. No BVDV positive animal on arrival and no |
| | exposure through the fence to an adjacent PI or PIR |
| | group. This test group was not exposed directly to a |
| | BVDV positive animal in the feedlot, but may have been |
| | exposed to BVDV prior to shipping or in the hospital |
| | area of that alley. |

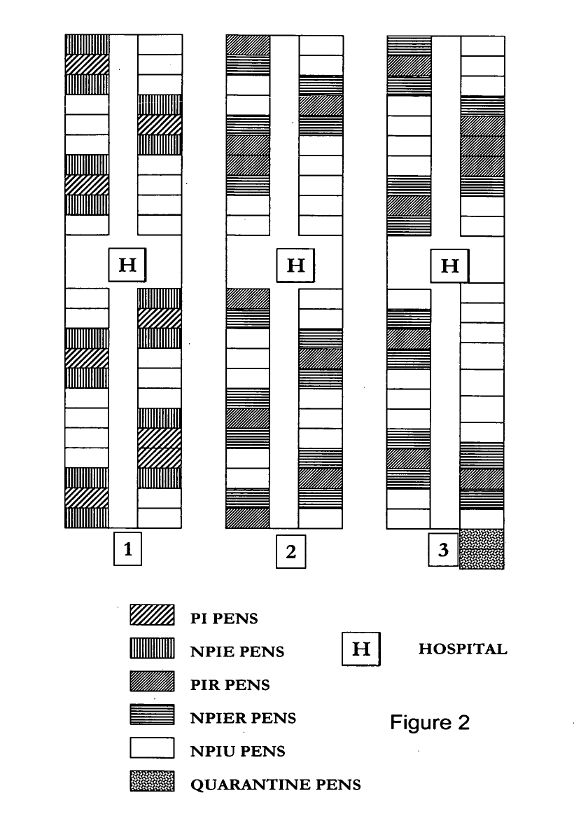

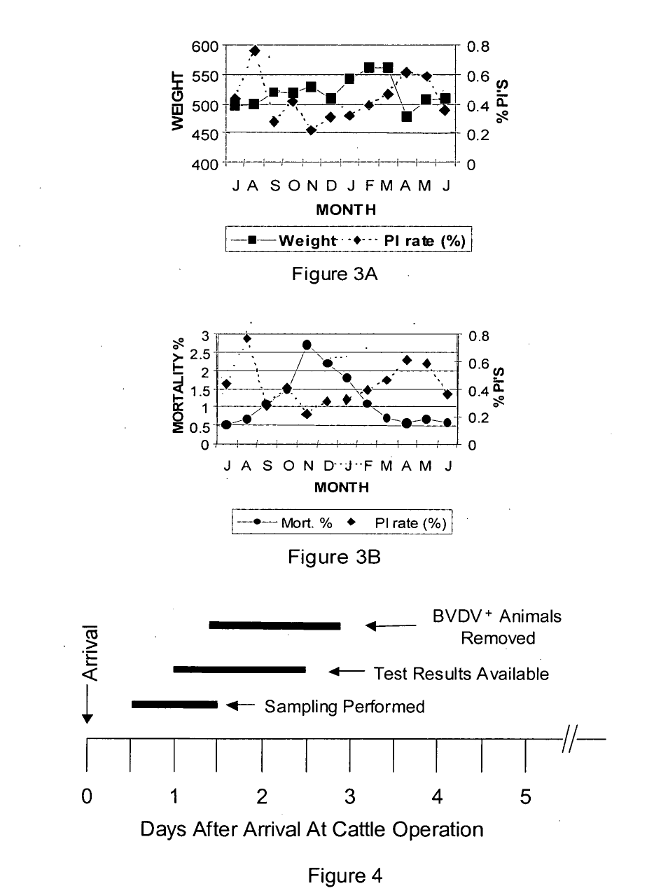

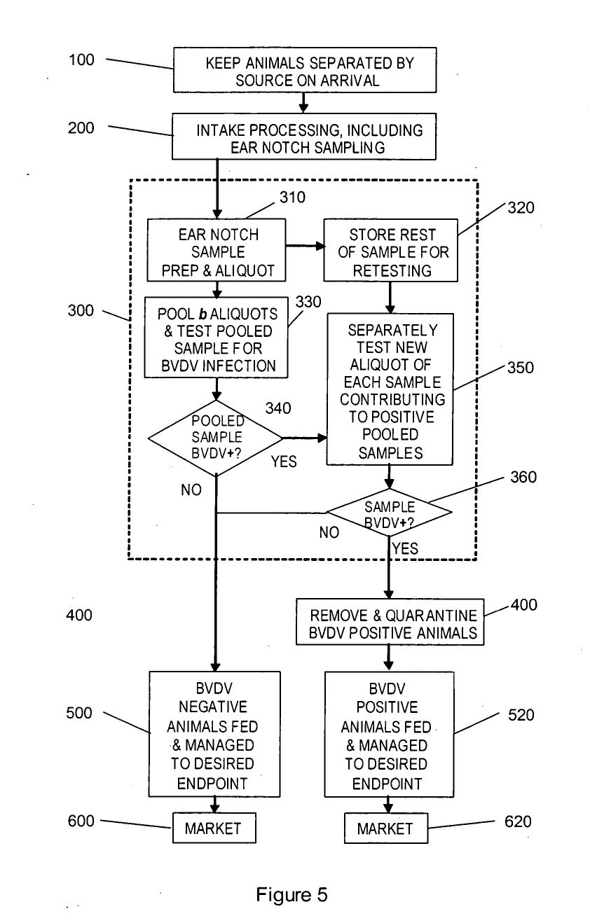

FIG. 2 is a schematic diagram illustrating the classification scheme underlying the designation of the PI, PIR, NPIE, NPIER and NPRU groups by position of the pens within the feedlot. In alley 1, the PI animals identified by the prophylactic screening were left in the pen with their arrival group, and such a pen is indicated as a PI pen in FIG. 2. In general, PI pens were flanked by pens that did not include BVDV positive animals; the flanking pens were designated as NPIE pens. NPIU pens were the remaining pens that neither contained a BVDV positive animal on arrival nor were adjacent to a PI pen.

In contrast, the PI animals in the pens of alleys 2 and 3 were removed to quarantine pens at the end of alley 3 after screening of all animals. The pens that had contained a PI animal that had been removed to quarantine were designated as PIR pens, and flanking pens that had not contained a PI animal were designated NPIER pens. The pens in alleys 2 and 3 that had neither contained a PI animal nor were adjacent to a pen that had contained a PI animal were designated NPIU pens. Each alley had a hospital area that was used to treat animals from pens in that alley.

All of the pens were filled and emptied at least twice during the course of the study, and thus the diagram illustrates the classification scheme, and does not necessarily show a snapshot of the distribution of animals within the feedlot at any given time. Some pens changed their status between NPIU, NPIE and NPIER over the course of the study if the status of an adjacent pen changed to or from PI or PIR.

The prevalence of PI animals in the study group by geographic source is summarized in Table 6. The geographic origin listed was the state that the buyer stated was the origin of most, but not necessarily all, of the cattle in a given group. The prevalence of PI animals varied from 0.16% to 2.0% depending on the nominal geographic origin.

Table 7 provides a summary of the prevalence of PI animals in the study group broken down by commercial source. This perspective, while not independent of the geographic source analysis, provides a more detailed view of smaller subsets of the study group. Again, there is a wide variation in PI prevalence from 0% (0/314) to 2% (8/403). Since the prevalence is highly variable from group to group as the cattle arrive in the feedlot, and will not be known until prophylactic screening, it is an important aspect of the livestock health management method of the present invention to keep the livestock separated by source on arrival to limit any further effect of PI animals on non-PI animals.

TABLE 6
PI Prevalence by Origin

| State | # Head | # PI's | Prevalence | # Pens | # Pos. Pens | Pen Rate |
|---|---|---|---|---|---|---|
| AR | 403 | 8 | 2.0% | 4 | 3 | 75% |
| NC | 851 | 4 | 0.47% | 11 | 4 | 36% |
| FL | 1930 | 3 | 0.16% | 18 | 3 | 17% |
| KY | 415 | 1 | 0.24% | 4 | 1 | 25% |
| MO | 1323 | 2 | 0.15% | 15 | 2 | 13% |
| MS | 756 | 1 | 0.13% | 8 | 1 | 13% |
| OK | 8184 | 42 | 0.51% | 88 | 36 | 41% |
| TN | 1227 | 7 | 0.57% | 14 | 7 | 50% |
| TX | 5691 | 15 | 0.26% | 67 | 15 | 22% |
| VA | 963 | 3 | 0.31% | 11 | 2 | 18% |

Variations in PI animal prevalence were also seen at different times in the course of the study. FIG. 3 is a graphic representation comparing the average PI prevalence and average weight-in over the course of a year. FIG. 3B is a graphic representation comparing the average PI prevalence and mortality over the course of a year. The PI prevalence did not appear to be significantly correlated to either weight-in or mortality over this period.

TABLE 7
PI Rate by Buyer

| | # | # | | # | | |
|---|---|---|---|---|---|---|
| Buyer | Bought | of PI's | Prevalence | of Pens | Pos. Pens | Pen Rate |
| 1 | 7480 | 38 | .51% | 80 | 32 | 40% |
| 2 | 1323 | 2 | .15% | 15 | 2 | 13% |
| 3 | 277 | 2 | .72% | 3 | 1 | 33% |
| 4 | 850 | 4 | .47% | 11 | 4 | 36% |
| 5 | 1604 | 2 | .12% | 15 | 2 | 13% |
| 6 | 3689 | 12 | .33% | 45 | 12 | 27% |
| 7 | 314 | 0 | 0.0% | 3 | 0 | 0% |
| 8 | 686 | 1 | .15% | 8 | 1 | 13% |
| 9 | 1227 | 7 | .57% | 14 | 7 | 50% |
| 10 | 756 | 1 | .13% | 8 | 1 | 13% |
| 11 | 102 | 1 | .98% | 1 | 1 | 100% |
| 12 | 326 | 1 | .31% | 3 | 1 | 33% |
| 13 | 221 | 1 | .45% | 2 | 1 | 50% |
| 14 | 403 | 8 | 2.0% | 4 | 3 | 75% |
| 15 | 1575 | 1 | .06% | 17 | 1 | 6% |
| 16 | 100 | 1 | 1.0% | 1 | 1 | 100% |
| 17 | 427 | 2 | .47% | 5 | 2 | 40% |
| 18 | 383 | 2 | .52% | 5 | 2 | 40% |

Animals were weighed and individually identified on arrival. Intake processing steps also included ear notch tissue sampling, 5-way viral vaccination, Pasteurella vaccination, deworming, clostridial vaccination and metaphylactic injection. Animals received the 5-way viral vaccination again ten days later. In general, standard starter yard management practices can include vaccinations, deworming, castration, administering growth promotants, giving prophylactic antimicrobials, tipping horns, adjusting cattle to high energy diets and alleviating any sickness that comes with commingling, transport and the other stresses of procurement. The calves in the study group were limit fed, and not implanted with growth promoters such as estradiol benzoate, zeranol, progesterone 17-beta-estradiol, trenbolone acetate and mixtures thereof.

All calves were ear-notched at initial processing and the samples were prophylactically screened using a commercially available BVDV antigen capture ELISA (ACE) test (Herdchek™, IDEXX, Westbrook, Me.). According to the ACE test manufacturer's directions, ear notch samples approximately 1 cm×1 cm were suspended in 2 ml of 0.1 M phosphate buffered saline pH 7.4 with 0.9% sodium chloride (PBS) in a plastic or glass vial and mixed by inversion or gentle vortexing. The ear notch was incubated at room temperature (18-25 degrees Celsius) for a minimum of ten minutes after mixing to form a tissue suspension. The tissue suspension was mixed by inversion or gentle vortexing before an aliquot was removed for testing. The ear notch sample in PBS can be refrigerated at 2-7 degrees Celsius for short-term storage (1-2 days) or frozen (−20 degrees Celsius are colder) for long-term storage.

Reducing the time that it takes to identify a subclinical PI animal and remove it from contact from its arrival group is an important aspect the method of the present invention. In general, the results showed that testing and removing any PI animal promptly can reduce the morbidity and mortality of the non-PI calves, as seen in the performance of the animals from PIR pens compared to the PI pens. Preliminary analysis of the data indicated that the benefit could be about $20.00/head (estimated range of $17.40-$24.30) for all animals placed in the starter yard. This benefit is for all animals placed in the yard not just those animals in the pens where the PI animal was removed. This total return is from improved health, less mortality and improved performance.

The overall prevalence of BVDV was 0.4% (4/1,000) and 30.8% of the pens that were placed on feed had at least one PI animal. However, the prevalence varied by source (buyer) and geographical origin, as shown in Tables 6 and 7, above.

Overall, 88 BVDV positive animals were identified in the prophylactic screening stage using the ACE test. Two of the 88 BVDV positive animals were subsequently found to be acutely infected. The prevalence of PI animals in this study was thus 86 out of 21,743 head, or 0.40%. Seventy-four pens were PI positive, a positive pen rate of 30.8%, with 61% of the feed yard population exposed. In a larger sample that included the cattle studied in Example 2, prophylactic screening of 305,667 head of cattle using the method of the present invention has identified 1232 positive animals, a 0.40% prevalence rate.

Twenty-two of the 86 PI animals (25.5%) died during the starter phase, in this study, about the first sixty days. The causes of death were: mucosal disease, 14/22 (64%); respiratory disease, 6/22 (27%); bloat, 1/22 (4.5%); other, 1/22 (4.5%). Four of 37 were railed from PI pens during the starter phase, at about 30-60 DOF of the study. Another forty three of the 86 PI animals were sold to slaughter or railed light at about 90 DOF.

Further testing determined the following virus subtypes in the PI animals: BVDV 1b, 77.9%; BVDV 1a, 11.6% and BVDV 2, 10.5%. Transmission of BVDV 1b from PI animals to both vaccinated and unvaccinated calves has been reported (Fulton, R. W., et al., Transmission of bovine viral diarrhea virus 1b to susceptible and vaccinated calves by exposure to persistently infected calves, Canadian J Vet Res 69: 161-169, 2005.

The results of analyses of the raw data and analyses of data sets from which outliers more than 3 standard deviations from the mean had been removed are presented in the Tables below. Analysis of variance procedures were conducted on all response variables using PROC MIXED in PC SAS Version 9 (SAS Institute, Cary, N.C.). Pair wise t-tests (LSMEANS statement with a DIFF option) were conducted to determine differences in status. A significance level of 0.05 was used for all comparisons.

As noted above, some pens changed their status between NPIU, NPIE and NPIER over the course of the study if the status of an adjacent pen changed to or from PI or PIR. For proposes of analysis of all pens, the pens that changed status were considered as belonging to the higher risk group if the animals in that pen spent the majority of their days on feed (DOF) classified the higher risk category. See Tables 8, 9, 10 and 11. In order to clarify trends, reduced data sets of the pens that did not change risk status, with and without outliers removed, were analyzed. See Tables 12, 13, 14 and 15.

Tables 8 & 10 and Tables 9 & 11 provide summaries of the effects of PI animal identification and removal on feedlot performance and on health, respectively, of all pens with and without the outliers.

TABLE 8
PERFORMANCE VARIABLES
ALL PENS - MAJORITY OF DOF AT HIGHEST RISK

| | | | WT OUT | WT GAIN | F/G DRY | ADG | COG |
|---|---|---|---|---|---|---|---|
| STATUS | PENS | WT IN | D IN | D IN | D IN | D IN | D IN |
| PI | 33 | 509^{a} | 584^{b} | 75^{d} | 18.88^{a} | 1.21^{c} | 2.86^{a} |
| PIR | 37 | 397^{a} | 582^{b} | 84^{cd} | 9.47^{b} | 1.30^{bc} | 1.40^{a} |
| NPIE | 25 | 517^{a} | 610^{ab} | 93^{bc} | 7.88^{b} | 1.44^{ab} | 1.13^{a} |
| NPIER | 35 | 523^{a} | 626^{a} | 102^{ab} | 7.12^{b} | 1.54^{a} | 1.05^{a} |
| NPIU | 84 | 517^{a} | 625^{a} | 108^{a} | 7.52^{b} | 1.59^{a} | 1.08^{a} |

Table 8 summarizes the performance data for all pens, each pen being classified in the highest risk category in which it spent the majority of the days on feed. The superscripts a, b, c and d are used in Tables 8-15 to link the groups that were not significantly different at the p≦0.05 level for that variable. Thus, the “a” superscripts for all “WT IN” data indicate that there was no significant difference in starting weight across all five groups. However, the weight out calculated with deads in (“WT OUT D IN”) was significantly greater for the NPIU or NPIER groups compared to the PI or the PIR groups. The weight gain calculated with deads in (“WT GAIN D IN”) for the NPIU, NPIER and NPIE groups was significantly greater that of the PI group. The conversion (“F/G DRY D IN”) was significantly worse in the PI group compared to all other groups, showing a significant improvement in the cattle in pens that previously had a PI animal that was removed. The average daily gain calculated with deads in (“ADG D IN”) was significantly greater for the NPIU, NPIER and NPIE groups compared to that of the PI group. The cost of gain, deads in (“COG D IN”) was significantly higher in the PI group compared to the NPI group in the study of Example 1. In this data set of Example 2, while the cost of gain of the PI group was higher than that of the other groups, the difference was not significant at the p≦0.05 level.

TABLE 9
HEALTH VARIABLES
ALL PENS - MAJORITY OF DOF AT HIGHEST RISK

| | | 1^{st} | 2^{nd} | 3^{rd} | | | | |
|---|---|---|---|---|---|---|---|---|
| | MORB | Relapse | Relapse | Relapse | RAIL | MORT | TX$/ | AVG # |
| STATUS | # | Rate | Rate | Rate | % | % | HD | OF TX'S |
| PI | 34.0^{a} | 46%^{a} | 58%^{a} | 22%^{a} | 4.6^{ab} | 3.6^{a} | 17.04^{a} | 1.79^{a} |
| PIR | 37.0^{a} | 46%^{a} | 51%^{a} | 27%^{a} | 5.0^{a} | 3.5^{a} | 15.70^{a} | 1.77^{a} |
| NPIE | 32.7^{a} | 45%^{a} | 49%^{a} | 29%^{a} | 5.4^{a} | 2.9^{ab} | 16.98^{a} | 1.75^{a} |
| NPIER | 32.0^{a} | 40%^{a} | 47%^{a} | 24%^{a} | 3.4^{ab} | 2.0^{b} | 15.51^{a} | 1.66^{a} |
| NPIU | 32.1^{a} | 41%^{a} | 51%^{a} | 32%^{a} | 3.2^{b} | 2.2^{b} | 15.74^{a} | 1.70^{a} |

Table 9 summarizes the results for all pens for eight health related variables. There was a significantly lower percent mortality (“MORT %”) in the NPIU and NPIER groups compared to the PI and PIR groups. The percent railers (“RAWL %”) was significantly lower in the NPIU group compared to the PIR and NPIE groups. There were no significant differences between the five groups in the other variables: percent morbidity (“MORB %”), first relapse rate (“1^{ST} RELAPSE RATE”), second relapse rate (“2^{ND} RELAPSE RATE”), third relapse rate (“3^{RD} RELAPSE RATE”), cost of treatment per head (“TX$/HD”) and the average number of treatments (“AVG # OF TX′S”).

TABLE 10
PERFORMANCE VARIABLES
ALL PENS - MAJORITY OF DOF AT HIGHEST RISK
(OUTLIERS REMOVED)

| | | | WT OUT | WT GAIN | F/G DRY | ADG | COG |
|---|---|---|---|---|---|---|---|
| STATUS | PENS | WT IN | D IN | D IN | D IN | D IN | D IN |
| PI | 33 | 506^{a} | 584^{b} | 78^{d} | 11.02^{a} | 1.25^{c} | 1.63^{a} |
| PIR | 35 | 497^{a} | 585^{b} | 87^{cd} | 8.27^{b} | 1.36^{bc} | 1.22^{b} |
| NPIE | 24 | 518^{a} | 614^{a} | 96^{bc} | 7.33^{b} | 1.48^{ab} | 1.03^{b} |
| NPIER | 34 | 520^{a} | 624^{a} | 104^{ab} | 6.96^{b} | 1.55^{a} | 0.97^{b} |
| NPIU | 82 | 518^{a} | 628^{a} | 111^{a} | 6.67^{b} | 1.62^{a} | 0.93^{b} |

Table 10 summarizes the performance data for all pens at the highest risk group for the majority of the days on feed, with outliers greater than three standard deviations from the mean removed. Compared to the data set of Table 8, there are two fewer PIR pens, one fewer NPIE pen, one fewer NPIR pen and are two fewer NPRU pens in this data set. Removal of the outliers lowers the average COG deads in for all groups. The difference in COG deads in between the PI group and all other groups is significant at the p≦0.05 level, indicating a significant favorable result of removing PI animals.

TABLE 11
HEALTH VARIABLES
ALL PENS - MAJORITY OF DOF AT HIGHEST RISK
(OUTLIERS REMOVED)

| | | 1^{st} | 2^{nd} | 3^{rd} | | | | |
|---|---|---|---|---|---|---|---|---|
| | MORB | Relapse | Relapse | Relapse | RAIL | MORT | TX$/ | AVG # |
| STATUS | # | Rate | Rate | Rate | % | % | HD | OF TX'S |
| PI | 34.0^{a} | 46%^{a} | 58%^{a} | 22%^{a} | 4.5^{a} | 3.5^{a} | 16.80^{a} | 1.79^{a} |
| PIR | 36.1^{a} | 43%^{a} | 49%^{a} | 26%^{a} | 4.7^{a} | 2.9^{a} | 15.84^{a} | 1.73^{a} |
| NPIE | 31.02 | 44%^{a} | 48%2 | 30%^{a} | 4.3^{a} | 2.7^{ab} | 16.26^{a} | 1.72 |
| NPIER | 31.7^{a} | 40%^{a} | 47%^{a} | 23%^{a} | 3.4^{a} | 2.1^{b} | 15.52^{a} | 1.65^{a} |
| NPIU | 31.2^{a} | 41%^{a} | 51%^{a} | 31%^{a} | 3.1^{a} | 2.0^{b} | 15.65^{a} | 1.69^{a} |

Table 11 summarizes the results for all pens for eight health related variables after removal of outliers, showing essentially the same situation in terms of significant difference as seen in Table 9, above. There was a significantly lower percent mortality (“MORT %”) in the NPRU and NPIER groups compared to the PI and PIR groups. There were no significant differences between the five groups in the other variables: percent morbidity (“MORB %”), first relapse rate (“1^{ST} RELAPSE RATE”), second relapse rate (“2^{ND} RELAPSE RATE”), third relapse rate (“3^{RD} RELAPSE RATE”), percent railers (“RAIL %”), cost of treatment per head (“TX$/HD”) and the average number of treatments (“AVG # OF TX′S”).

TABLE 12
PERFORMANCE VARIABLES
PENS WITH NO STATUS CHANGE

| | | | WT OUT | WT GAIN | F/G DRY | ADG | COG |
|---|---|---|---|---|---|---|---|
| STATUS | PENS | WT IN | D IN | D IN | D IN | D IN | D IN |
| PI | 33 | 506^{a} | 584^{b} | 75^{b} | 18.88^{a} | 1.21^{c} | 2.86^{a} |
| PIR | 37 | 497^{a} | 582^{b} | 84^{b} | 9.47^{a} | 1.30^{bc} | 1.40^{b} |
| NPIE | 17 | 529^{a} | 622^{a} | 93^{ab} | 7.27^{a} | 1.49^{ab} | 1.02^{b} |
| NPIER | 16 | 533^{a} | 638^{a} | 105^{a} | 6.57^{a} | 1.61^{a} | 0.91^{b} |
| NPIU | 64 | 514^{a} | 624^{a} | 110^{a} | 6.78^{a} | 1.63^{a} | 0.95^{b} |

Table 12 summarizes the performance data for all pens with no status change.

Compared to the data set of Table 8, there are eight fewer NPIE pens, nineteen fewer NPIER pens and are twenty fewer NPIU pens in this data set. Removal of the pens that changed status lowers the average COG deads in for groups NPIE, NPIER and NPIU.

The difference in COG deads in between the PI group and all other groups is significant at the p≦0.05 level, again indicating a significant favorable result of removing PI animals. Weight out, deads in is significantly lower for the PI and PIR groups compared to the NPIE, NPIER and NPIU groups. Weight gain, deads in, is significantly lower for the PI and PIR groups compared to the NPIER and NPIU groups.

TABLE 13
HEALTH VARIABLES
PENS WITH NO STATUS CHANGE

| | | 1^{st} | 2^{nd} | 3^{rd} | | | | |
|---|---|---|---|---|---|---|---|---|
| | MORB | Relapse | Relapse | Relapse | RAIL | MORT | TX$/ | AVG # |
| STATUS | % | Rate | Rate | Rate | % | % | HD | OF TX'S |
| PI | 34.0^{ab} | 46%^{a} | 58%^{a} | 22%^{a} | 4.6^{a} | 3.6^{a} | 17.04^{a} | 1.79^{a} |
| PIR | 37.0^{a} | 46%^{a} | 51%^{a} | 27%^{a} | 5.0^{a} | 3.5^{a} | 15.69^{a} | 1.77^{a} |
| NPIE | 29.2^{bc} | 45%^{ab} | 46%^{a} | 30%^{a} | 3.6^{ab} | 2.4^{ab} | 16.45^{a} | 1.72^{abc} |
| NPIER | 24.8^{c} | 35%^{c} | 49%^{a} | 24%^{a} | 2.7^{b} | 1.3^{b} | 14.30^{a} | 1.58^{c} |
| NPIU | 29.0^{bc} | 40%^{bc} | 48%^{a} | 30%^{a} | 2.8^{b} | 1.7^{b} | 15.65^{a} | 1.66^{b} |

Table 13 summarizes the results for eight health related variables for the pens with no status change, showing a somewhat different situation in terms of significant difference from that in Tables 9 and 11, above. As in Tables 9 and 11, there was a significantly lower percent mortality (“MORT %”) in the NPIU and NPIER groups compared to the PI and PIR groups. However, in this data set there was a significantly lower percent morbidity rate in the NPIER group compared to the PI and PIR groups, and a significant lower first relapse rate, and average number of treatments in the NPIU and NPIER groups compared to the PI and PIR groups. There was a significantly lower average percent railers in the NPIU and NPIER groups compared to the PI and PIR groups. There were no significant differences between the five groups in the other variables: second relapse rate, third relapse rate and cost of treatment per head.

TABLE 14
PERFORMANCE VARIABLES
PENS WITH NO STATUS CHANGE
(OUTLIERS REMOVED)

| | | | WT OUT | WT GAIN | F/G DRY | ADG | COG |
|---|---|---|---|---|---|---|---|
| STATUS | PENS | WT IN | D IN | D IN | D IN | D IN | D IN |
| PI | 32 | 506^{a} | 584^{b} | 78^{c} | 11.02^{a} | 1.25^{c} | 1.63^{a} |
| PIR | 35 | 497^{a} | 585^{b} | 87^{bc} | 8.27^{ab} | 1.36^{bc} | 1.22^{ab} |
| NPIE | 17 | 529^{a} | 622^{a} | 93^{bc} | 7.27^{b} | 1.49^{ab} | 1.02^{b} |
| NPIER | 16 | 533^{a} | 638^{a} | 105^{ab} | 6.57^{b} | 1.61^{a} | 0.91^{b} |
| NPIU | 63 | 514^{a} | 625^{a} | 111^{a} | 6.44^{b} | 1.65^{a} | 0.89^{b} |

Table 14 summarizes the performance data for all pens with no status change with outliers greater than three standard deviations from the mean removed. Compared to the data set of Table 12, there is one fewer PI pen, two fewer PIR pens, and one fewer NPIU pen in this data set. Removal of the outliers lowers the average COG deads in for groups PI and PIR. The difference in COG deads in between the PI group and groups NPIE, NPIER and NPIU is significant at the p≦0.05 level, again indicating a significant favorable result of removing PI animals. In addition, weight out, deads in, is significantly lower for the PI and PIR groups compared to the NPIE, NPIER and NPIU groups. Conversion, deads in, is significantly poorer for the PI group compared to the NPIE, NPIER and NPIU groups. Weight gain, deads in, for the NPIU group is more than 40% more than that of the PI group and about 27% greater than that of the PIR group.

TABLE 15
HEALTH VARIABLES
PENS WITH NO STATUS CHANGE
(OUTLIERS REMOVED)

| | | 1^{st} | 2^{nd} | 3^{rd} | | | | |
|---|---|---|---|---|---|---|---|---|
| | MORB | Relapse | Relapse | Relapse | RAIL | MORT | TX$/ | AVG # |
| STATUS | % | Rate | Rate | Rate | % | % | HD | OF TX'S |
| PI | 34.0^{ab} | 46%^{a} | 59%^{a} | 22%^{a} | 4.5^{a} | 3.5^{a} | 16.80^{a} | 1.79^{a} |
| PIR | 36.0^{a} | 43%^{ab} | 49%^{a} | 26%^{a} | 4.7^{a} | 2.9^{a} | 15.84^{a} | 1.73^{a} |
| NPIE | 29.2^{bc} | 45%^{ab} | 46%^{a} | 30%^{a} | 3.6^{ab} | 2.4^{ab} | 16.45^{a} | 1.72^{a} |
| NPIER | 24.8^{c} | 35%^{c} | 49%^{a} | 24%^{a} | 2.7^{b} | 1.3^{b} | 14.30^{a} | 1.58^{a} |
| NPIU | 28.5^{bc} | 39%^{bc} | 48%^{a} | 30%^{a} | 2.7^{b} | 1.6^{b} | 15.46^{a} | 1.65^{a} |

Table 15 summarizes the results for all pens for eight health related variables after removal of outliers, showing a somewhat different situation in terms of significant difference from that in Tables 9 and 11, above. As in Tables 9, 11 and 13, there was a significantly lower percent mortality (“MORT %”) in the NPIU and NPIER groups compared to the PI and PIR groups. As in the Table 13 data set there was a significantly lower percent morbidity rate in the NPIER group compared to the PI and PIR groups, and a significant lower first relapse rate and percent railers in the NPIU and NPIER groups compared to the PI and PIR groups. There were no significant differences between the five groups in the other variables: second relapse rate, third relapse rate, cost of treatment per head and average number of treatments.

The analysis of the data set from pens that did not change status during the study, with outliers removed, was further pursued using the data set of Tables 14 and 15. A first comparison, between NPIU and PI+NPIE pens, examines the effects of lack of direct or indirect contact with a PI animal to the effects seen in animals that were in direct or indirect contact with a PI animal through the course of the study. This is a slightly different grouping than the comparison of NPI and PI pens in Example 1 (Tables 1 and 2), where the animals in the pens adjacent to the PI pens were lumped in the NPI group, instead of having a separate class, NPIE. In Example 1, the NPI group had a significantly greater weight gain than the PI group. In Example 2, the weight gain, deads in, was NPIU>NPIER>NPIE>PIR>PI (Table 14), and the difference NPRU v. PI+NPIE was significant (Table 16). Furthermore, the difference in ADG, also was ranked NPIU>NPIER>NPIE>PIR>PI (Table 14), and the difference NPIU v. PI+NPIE was significant (Table 16). For comparison, the difference in ADG between NPI and PI groups in Example 1 had p=0.07. In summary, either direct or indirect with a PI animal left in a pen had significant effects on weight out (deads in), weight gain, ADG, COG (deads out), percent railers and percent mortality when compared to animals that were not exposed to a PI animal in transit or in the feedlot.

A second comparison was between PI and PIR pens to examine whether the removal of a PI animal has a beneficial effect on the remaining cattle. The results are shown in Table 16, below. The differences between these groups that were most significant were in conversion (F/G DRY, deads in or deads out) and cost of gain (COG, deads in or deads out). However, several differences that were significant at the p≦0.05 level for the NPIU v. PI+NPIE comparison were not significant at that level for the PI v. PIR comparison, e.g. weight out (deads in), weight gain (deads out or in), RAWL % and percent mortality. Thus, while some of the effects of direct contact with a PI animals can be reduced by removal of the PI, other effects remain.

A third comparison, between PIR and NPIER+NPIU pens examines the effects of direct contact with a PI animal that was removed within a few days of arrival compared to pens that did not have direct contact with a PI animal. The results are shown in Table 16, below. The differences between these groups that were most significant were in weight in, weight out, weight gain, ADG, 1^{st} relapse rate, RAIL % and MORT %. Thus, consistent with the results of the PI v. PIR comparison, some of the effects of direct contact with a PI can be reduced by removal of the PI animal, other variables are still significantly different from the values seen in pens that have not experienced direct exposure to a PI animal.

TABLE 16
Comparison of Treatment Groups

| | P values | | |
|---|---|---|---|
| | NPIU v PI + | | PIR v NPIER + |
| Variable | NPIE | PI v PIR | NPIU |
| WT IN | 0.62 | 0.40 | 0.02 |
| WT OUT Deads In | 0.004 | 0.97 | <0.0001 |
| WT GAIN Deads In | <0.0001 | 0.18 | 0.001 |
| WT GAIN Deads Out | 0.0002 | 0.23 | 0.04 |
| F/G DRY Deads In | 0.07 | 0.06 | 0.56 |
| F/G DRY Deads Out | 0.003 | 0.02 | 0.63 |
| ADG Deads In | 0.0001 | 0.21 | 0.001 |
| ADG Deads Out | 0.0002 | 0.22 | 0.01 |
| COG Deads In | 0.08 | 0.07 | 0.56 |
| COG Deads Out | 0.003 | 0.04 | 0.27 |
| MORB % | 0.63 | 0.40 | 0.07 |
| 1^{st} Relapse Rate | 0.08 | 0.64 | 0.05 |
| 2^{nd} Relapse Rate | 0.42 | 0.06 | 0.60 |
| 3^{rd} Relapse Rate | 0.18 | 0.55 | 0.88 |
| RAIL % | 0.01 | 0.80 | 0.03 |
| MORT % | 0.04 | 0.30 | 0.02 |
| TX Cost | 0.14 | 0.40 | 0.95 |
| Avg. No. TX | 0.19 | 0.33 | 0.10 |

Tables 17 and 18 provide similar information about the comparison of two biological groups PI+NPIE and PIR+NPIER. The difference between the groups was significant at the p≦0.05 level only for the first relapse variable.

TABLE 17
RESULTS BY BIOLOGIC GROUPS

| | | | | WT | WT | | | | | | |
|---|---|---|---|---|---|---|---|---|---|---|---|
| | | WT OUT | WT OUT | GAIN | GAIN | F/G | F/G | ADG | ADG | COG | COG |
| TREATMENT | Weight | DEADS | DEADS | DEADS | DEADS | DEADS | DEADS | DEADS | DEADS | DEADS | DEADS |
| GROUP | In | IN | OUT | IN | OUT | IN | OUT | IN | OUT | IN | OUT |
| PI & NPIE | 514 | 597 | 616 | 83 | 102 | 9.72 | 6.48 | 1.33 | 1.60 | 1.42 | 0.93 |
| PIR & NPIER | 508 | 601 | 617 | 93 | 109 | 7.74 | 6.13 | 1.44 | 1.66 | 1.12 | 0.88 |
| P-value | 0.79 | 0.46 | 0.76 | 0.09 | 0.31 | 0.17 | 0.08 | 0.11 | 0.29 | 0.18 | 0.09 |

TABLE 18
MORBIDITY & MORTALITY BY BIOLOGIC GROUPS

| TREATMENT | MORB | 1ST | 2ND | 3RD | RAIL | MORT | TX COST/ | # OF |
|---|---|---|---|---|---|---|---|---|
| GROUP | % | RELAPSE | RELAPSE | RELAPSE | % | % | HD PLACED | TREATMENTS |
| PI & NPIE | 32 | 46 | 54 | 25 | 4.2 | 3.1 | 16.68 | 1.77 |
| PIR & NPIER | 33 | 41 | 41 | 49 | 4.1 | 2.4 | 15.36 | 1.68 |
| P-value | 0.73 | 0.03 | 0.47 | 0.81 | 0.63 | 0.08 | 0.11 | 0.07 |

The data for NPIU pens in alley 1 were compared to those for NPIU pens in alleys 2 and 3 to look for possible exposure to PI animals due to the presence of hospital areas in alley 1. There was a hospital area in each alley to prevent further contamination of cattle in alleys 2 and 3 when treating PI animals in alley 1. The results are presented in Tables 19 and 20.

TABLE 19
HOSPITAL EFFECT ON RESULTS

| | | | WT OUT | | WT GAIN/HD | | F/G DRY | | ADG | | COG | COG |
|---|---|---|---|---|---|---|---|---|---|---|---|---|
| PENS | COUNT | WT IN | D IN | D OUT | D IN | D OUT | DEADS IN | DEADS OUT | D IN | D OUT | D IN | D OUT |

| | | | | | | | | | | | | | |
|---|---|---|---|---|---|---|---|---|---|---|---|---|---|
| NPIU ALLEY 1 | 17 | 1541 | 530 | 653 | 670 | 122 | 140 | 6.65 | 5.83 | 1.67 | 1.87 | 0.908 | 0.798 |
| NPIU ALLEY | 46 | 4174 | 508 | 615 | 623 | 107 | 115 | 6.36 | 5.90 | 1.64 | 1.75 | 0.879 | 0.819 |
| 2 & 3 | | | | | | | | | | | | | |

The data on performance variables (Table 19) are difficult to interpret in early analysis due to the lower average weight in of the NPIU pens in alleys 2 and 3.

TABLE 20
HOSPITAL EFFECT ON MORBIDITY & MORTALITY

| | | 1ST | 2ND | 3RD | | | | | | |
|---|---|---|---|---|---|---|---|---|---|---|
| | MORB | RELAPSE | RELAPSE | RELAPSE | RAIL | MORT | TX COST/ | AVG # | brd | brd |
| | % | RATE | RATE | RATE | % | % | HD PLACED | OF TX'S | mort % | morb % |
| NPIU ALLEY 1 | 30.91 | 44.70% | 56.95% | 29.99% | 3.18 | 2.59 | 17.72 | 1.78 | 2.01% | 29.01% |
| NPIU ALLEY 2 & 3 | 27.57 | 37.54% | 45.00% | 30.04% | 2.57 | 1.26 | 14.62 | 1.61 | 1.05% | 26.57% |

The data on health variables (Table 20) show the NPIU pens in alleys 2 and 3 lower than those in alley 1 in percent morbidity, percent railers, percent mortality, first relapse rate second relapse rate and cost of treatment. Mortality and morbidity due to bovine respiratory disease (BRD) were also lower for the NPIU pens in alleys 2 and 3 compared to those in alley 1.

The summary of the effects of fence exposure to PI animals compared to water tank exposure to PI animals on results and health is presented in Table 21. While the sample size was small, there was an indication that water tank exposure was associated with lower weight gain, lower ADG and higher COG. The pens having water tank exposure to PI animals tended to have higher morbidity and mortality that the pens having fence exposure to PI animals.

TABLE 21
FENCE VS WATER TANK EXPOSURE
EFFECT ON RESULTS

| | | WT | WT OUT | | WT GAIN/HD | | F/G | | ADG | | COG | COG |
|---|---|---|---|---|---|---|---|---|---|---|---|---|
| | PENS | IN | D IN | D OUT | D IN | D OUT | D IN | D OUT | D IN | D OUT | D IN | D OUT |
| F | 5 | 525 | 624 | 644 | 99 | 119 | 7.18 | 6.09 | 1.49 | 1.75 | 0.951 | 0.813 |
| W | 7 | 528 | 617 | 632 | 89 | 104 | 7.55 | 6.38 | 1.42 | 1.63 | 1.097 | 0.934 |

FENCE VS WATER TANK EXPOSURE
EFFECT ON MORBIDITY & MORTALITY

| | | | BRD | 1ST | | | | | |
|---|---|---|---|---|---|---|---|---|---|
| | | MORB | MORB | RELAPSE | RAIL | MORT | BRD Mort | TX COST/ | AVG # |
| | PENS | % | % | RATE | % | % | % | HD PLACED | OF TX'S |
| F | 5 | 24.91 | 23.37 | 41.46% | 2.37 | 2.90 | 1.73 | 14.72 | 1.68 |
| W | 7 | 32.45 | 32.45 | 48.31% | 3.64 | 2.39 | 2.26 | 17.40 | 1.74 |

### Summary of Performance Variables

The study of Example 1 showed that the absence of a PI animal produced differences significant at the p≦0.05 level in weight gain, conversion and cost of gain, with a difference in average daily gain that was significant at the 0.07 level (Table 1). The results of the study of Example 2 confirmed these findings and indicated that some variables may be more sensitive to the presence of a PI animal than others.

Generally, no difference was found between PI pens and PIR pens for the variables weight out, deads in, weight gain deads in and average daily gain. Weight out, deads in, showed a difference between significant at the p≦0.05 level for the comparison of PI pens versus NPIU, NPIER or NPIE pens and no significant difference between PI pens and PIR pens (Tables 8, 10, 12, 14 and 16). Similarly, no difference at the p≦0.05 level was found between PI pens and PIR pens for weight gain, deads in, (Tables 8, 10, 12, 14 and 16) but a significant difference was seen between PI pens and NPIU or NPIER pens. Average daily gain was significantly different at the p≦0.05 level for comparisons of PI pens versus NPIU, NPIER or NPIE pens (Tables 8, 10, 12 and 14), but no difference at the p≦0.05 level was found between PI pens and PIR pens (Tables 8, 10, 12, 14 and 16).

The results of the study of Example 2 for two other variables, conversion (F/G DRY deads in) and cost of gain, deads in, showed an indication of a difference between PI and PIR groups near, if not at, the p≦0.05 level. Conversion showed a difference significant at the p≦0.05 level for the comparison of PI pens versus NPIU, NPIER or NPIE pens (Tables 8, 10 and 14) and an indication of a significant difference between PI pens and PIR pens in Tables 8, 10 and at p=0.06 in Table 16. Cost of gain, deads in, showed a difference significant at the p≦0.05 level for the comparison of PI pens versus NPIU, NPIER or NPIE pens (Tables 10, 12 and 14) and an indication of a significant difference between PI pens and PIR pens in Tables 10 and 12 and at p=0.07 in Table 16.

### Summary of Health Variables

The study of Example 1 showed no differences significant at the p≦0.05 level in percent morbidity, percent mortality, percent railers, first relapse rate, second relapse rate, average number of treatments or cost of treatment per head (Table 2). The results of the study of Example 2 showed a difference in percent mortality that was significant at the p≦0.05 level for the comparison of either PI pens or PIR pens versus NPIU pens or NPIER pens, but no difference at the p≦0.05 level was found between PI pens and PIR pens (Tables 9, 11, 13, 15 and 16). A difference in percent railers was seen between PI pens and other pens that was generally not significant at the p≦0.05 level (compare Table 15 to Tables 9, 11, 13, and 16). No differences significant at the p≦0.05 level were found in percent morbidity, second relapse rate, average number of treatments or cost of treatment per head. A difference between PI pens and NPIU pens in first relapse rate was significant at the p≦0.05 level for the data set of Table 15, for the difference between PIR pens versus NPIU plus NPIER pens in the same data set (Table 16) and for the biological grouping of PI plus NPIE versus PIR plus NPIER (Table 17).

FIG. 4 is a schematic diagram of the first days after the arrival of the livestock at the cattle operation illustrating for the study of Example 2 the timing of ear notch sampling, reporting of the BVDV antigen capture ELISA test results and removal of the individual BVDV positive individual animals. Depending on the time of day and the day of the week of the arrival of the livestock, the ear notch sampling of the identified animals in performed between about 12 hours and 36 hours after arrival. Generally, the tests were performed soon after the samples were taken, and the test results were available about 12 hours later, i.e., at about 1 to 2.5 days after arrival. Typically, an identified BVDV positive animal was removed from its arrival group about 1.5 to 3 days after arrival, routinely within 48 hours after arrival. Typically, if test results were available in the afternoon or evening, the identified BVDV positive animal was removed from its arrival group before noon of the following day.

### EXAMPLE 3

### Modifications of Testing Strategies to Support Prompt Prophylactic Screening of All Animals

There are three primarily economic constraints for establishing a strategy of prophylactic screening in the practice of the present invention. First, the prevalence of the PI animals must be low, and the economic advantage of separating the PI animals from the general population must be significant. The economic advantage of removing the PI animals is established by the results of the studies that are described in detail below. Second, the time involved in the prophylactic screening testing must be as short as possible to reduce the exposure of the rest of the livestock to subclinical PI animals. Third, the test chosen must have a minimal false positive rate, to minimize the cost of unnecessary retesting, as well as a minimal false negative rate. In view of these constraints, not all tests that are suitable for diagnosis of a suspected PI animal are acceptable for prophylactic screening.

Diagnostic tests to detect the BVD virus include virus isolation with a microplate immunoperoxidase detection system, antigen capture ELISA (ACE), immunohistochemistry (IHC) and nuclear acid detection using polymerase chain reaction (PCR). See Drew, T., “Bovine Viral Diarrhoea,” Chapter 2.10.6 in Manual of Diagnostic Tests and Vaccines for Terrestrial Animals, 5^{th} Ed., 2004, World Organisation For Animal Health (Office International des Epizooties, OIE), http://www.oie.int/eng/normes/mmanual/A_{—}00132.htm.

The standard virus isolation protocol involves incubating the sample with tissue culture cells for four-five days before testing for the presence of the virus (Drew, 2004). This inherent delay of the virus isolation test is inappropriate for a prophylatic screening test to permit prompt identification and removal of PI animals from the rest of the cattle.

Similarly, the immunohistochemistry test of a skin biopsy specimen is also labor intensive and requires a turn-around time of 7-9 days before the results are available. The immunohistochemistry test was used to identify BVDV positive animals in the study of Example 1. However, during the wait for the test results any PI animal is still in its arrival group spreading BVDV.

An alternative test used to diagnose PI animals is the polymerase chain reaction test or PCR. This test can also be performed on a skin sample and is highly sensitive. However, the PCR test requires at least 96 hours for results and has a greater number of both false positive and false negative results compared to the ACE test. The PCR test is more expensive than the ACE test.

The ACE test, which is the testing method used in the study of Examples 2, can be completed in less than one day, preferably in about twelve hours, after receiving samples, thus reducing the delay in removing PI animals and minimizing the exposure of other livestock to the pathogen. This test has the quickest turn-around time of all the tests available for PI testing. However, the challenge of prophylactic screening of all livestock entering a feedlot is structuring the test strategy so that the costs of testing are less than the economic benefits produced.

In preferred embodiments, an improved screening and testing has been developed. It has been found that a cost-effective way of using the ACE test for the purpose of prophylactic screening is to perform the testing in two stages. In such embodiments, all calves were ear-notched at initial processing and the samples were prophylactically screened using a modification of a commercially available BVDV antigen capture ELISA (ACE) test (Herdchek™, IDEXX, Westbrook, Me.) to accommodate first round testing using pooled aliquots of samples from several animals as described in detail below. All aliquots that were combined in BVDV positive pooled sample were retested separately in order to identify the individual PI calves.

In the first stage aliquots of samples from several animals are tested as a pooled sample in one well. The pooled sample approach also has the advantage of increasing the speed at which samples can be sampled, since at a PI prevalence rate of 0.3-0.4 percent or less, more than 99% of the animals are expected to be BVDV negative. In the second stage, aliquots of the samples contributing to the few positive pooled samples can be retested separately to identify the individual subclinical PI animals.

This testing strategy differs from other pool and test protocols in favoring a prompt turn around time over optimizing of test cost, distinguishing for diagnostic purposes between PI and acutely infected animals or improving the accuracy of a prevalence estimate. In preferred embodiments, the testing strategy involves one stage of testing pooled samples followed by one stage of testing individual samples, with the entire testing procedure from sample preparation through reporting test results requiring less than a day after receiving samples, preferably about twelve hours after receiving samples.

While statistical analyses based on binomial distributions and Monte Carlo simulations have been used to optimize pooled sample testing, the basic principles can be shown by a simple algebraic discussion. The maximum number of tests y that would be required to screen x samples is given by Equation 1,
Y=z₁+z₂ (Equation 1)
where z₁, the maximum number of tests of pooled samples, is the integer that is greater or equal to x/b, where x is the number of samples and b is the number of aliquots from individual samples that are combined to make a pooled sample, and z₂, the maximum number of tests of individual samples, is the integer that is greater or equal to abx, where a is the estimated prevalence of positive animals in the screened population, x is the number of samples and b is the number of aliquots from individual samples that are combined to make a pooled sample. With an estimated prevalence of 0.4%, four animals per thousand would be expected to be BVD positive, with as many as four positive pooled samples that would require retesting, although as few as one pooled sample might test positive and require retesting. Examples of values are illustrated in Table 22, below.

Preferably, aliquots of 100/b μl are taken from each sample vial, where b is the number of animals to be tested in each pooled sample. Low values of b provide minimal cost advantage, but high values of b may reduce the sensitivity of the test due to increased dilution of antigen in the pooled samples and reduce any cost advantage gained in pooling samples by increasing the number of tests that must be performed in the second round of testing. In general, the appropriate value of b is determined by the prevalence of PI animals, the economic value of prompt isolation of PI animals and the unit cost of the test. In preferred embodiments, in prophylactic screening for BVDV using the ACE test, b is suitably in the range 2-10. In certain preferred embodiments, b is 6-9. In one preferred embodiment, b is 8, and 12.5 μl aliquots from each of eight samples are pooled to yield a 100 μl test sample.

As expected, the vast majority of the pooled samples were found to be BVDV negative. When a pooled sample tests positive, at least one of the aliquots contributing to the pooled sample must be BVDV positive. Another aliquot is taken from each of the b samples that contributed to that pool and tested separately. The animal that is identified as BVDV positive is immediately removed from its arrival group and quarantined. Because of short turn-around time of the ACE test, it is possible to quarantine PI animals about two days after the samples are collected. Optimally, analysis and sampling processes overlap to minimize the exposure of the non-PI animals in an arrival group.

TABLE 22
Estimated Number of Tests, y, for Screening 1000 Samples, x

| Prevalence | Pooling | | | |
|---|---|---|---|---|
| (a) | Factor (b) | x/b | abx | y |
| 0.003 | 2 | 500 | 6 | 506 |
| 0.003 | 3 | 333.3 | 9 | 343 |
| 0.003 | 4 | 250 | 12 | 262 |
| 0.003 | 5 | 200 | 15 | 215 |
| 0.003 | 6 | 166.7 | 18 | 185 |
| 0.003 | 7 | 142.9 | 21 | 164 |
| 0.003 | 8 | 125 | 24 | 149 |
| 0.003 | 9 | 111.1 | 27 | 139 |
| 0.003 | 10 | 100 | 30 | 130 |
| 0.001 | 5 | 200 | 5 | 205 |
| 0.004 | 5 | 200 | 20 | 220 |
| 0.007 | 5 | 200 | 35 | 235 |
| 0.01 | 5 | 200 | 50 | 250 |
| 0.001 | 8 | 125 | 8 | 133 |
| 0.004 | 8 | 125 | 32 | 157 |
| 0.007 | 8 | 125 | 56 | 181 |
| 0.01 | 8 | 125 | 80 | 205 |
| 0.001 | 10 | 100 | 10 | 110 |
| 0.003 | 10 | 100 | 30 | 130 |
| 0.004 | 10 | 100 | 40 | 140 |
| 0.007 | 10 | 100 | 70 | 170 |
| 0.01 | 10 | 100 | 100 | 200 |

Pooled sample testing regimes have been described before, but have been based on complex statistical models, provided protocols having multiple steps of pooling, dividing and re-pooling samples and provided inconsistent teachings as to preferred number of samples to pool. Munoz-Zanzi, C. A., et al., Pooled-sample testing as a herd-screening tool for detection of bovine viral diarrhea virus persistently infected cattle, J Vet Diagn Invest. 12(3): 195-203, 2000, disclosed that Monte Carlo simulations showed that the protocol associated with the least cost per cow involved an initial testing of pools followed by re-pooling and testing of positive pools, with intermediate splitting and testing of positive pools before testing aliquots of the individual samples that contributed to a positive pool. In some test strategies optimized to reduce the cost per cow of screening for BVDV, the samples from individual animals were not tested separately until the fourth round of testing. In other optimized pooled sample test strategies, positive pools are retested before splitting (Kennedy, N. L., Hierarchical screening with retesting in a low prevalence population, Sankhy a̅: The Indian Journal of Statistics, 66: 779-790, 2004). Larson et al. have proposed a two-stage test strategy of PCR testing of pooled blood samples followed by immunohistochemical staining of skin biopsy specimens from animals that had contributed to a pooled blood sample that had tested positive by PCR test (Larson, R. L., et al., Economic costs associated with two testing strategies for screening feeder calves for persistent infection with bovine viral diarrhea virus, J Am Vet Med Assoc 226:249-254, 2005). The proposed strategy of Larson et al., 2005, uses immunohistochemistry of a skin sample, which requires 7-9 days, after PCR testing of pooled blood samples, which itself would take several days. Larson et al. could not directly measure in their the economic cost of a PI animal in a group of feeder cattle, and did not consider the value of the time spent in the screening test protocol.

FIG. 5 is a schematic diagram of a preferred embodiment of the livestock management method of the present invention, showing the steps of keeping the livestock separated by source on arrival in the feedlot 100; performing intake processing, including ear notch sampling 200; performing prophylactic screening for BVDV infection 300; removing BVDV positive animals to quarantine 400; and feeding and managing BVDV negative animals 500 and BVDV positive animals 520 separately. In certain preferred embodiments, the method also includes the step of selling the animals 600, 620.

In preferred embodiments, the step of performing prophylactic screening for BVDV infection 300 includes the steps of ear notch sample preparation and aliquoting 310, reserving and storing the balance of each sample for availability for retesting 320, pooling aliquots from b samples, and testing the pooled sample for BVDV infection 330. If a pooled sample is BVDV negative, the corresponding animals are retained in their arrival group to be fed and managed 500 to the desired endpoint. If a pooled sample is BVDV positive, an aliquot from each sample that contributed to that pooled sample is retested individually 350. The animals corresponding to the BVDV positive aliquots at this second stage of screening are removed from contact with their arrival group, quarantined 400. The quarantined BVDV positive animals are fed and managed 520 to the desired endpoint.

In preferred embodiments, the prophylactic screening for BVDV infection 300 is performed in 1-2 days, more preferably in about twelve hours after ear notch samples are taken. Preferably, BVDV positive animals are removed from their arrival group and quarantined within about 1-2 days after test results are available. Animals upon arrival are under stress from shipping and have increased susceptibility to infections. Pathogen positive animals should be identified promptly through prophylactic screening and removed from contact with pathogen negative animals.

As a practical matter, animals that are found to be BVDV positive after the prophylactic screening step 300 are considered PI, even though proper diagnostic procedure involves resampling and retesting about thirty days after the initial test (if using viral isolation) to be able to distinguish acutely infected animals from PI animals. In general, it would be expected that less than about 10% of the BVDV positive animals would be shown to be acutely infected instead of PI on resampling and retesting. In the study described in Example 2 above, only two of 88 animals (2.2%) found to be BVDV positive in the initial prophylactic screening were determined to be acutely infected on further testing.

The results show that it is very cost-effective to test for and remove any PI animal from the population. Prior to this time PI testing, was mainly done when circumstances indicated that BVDV or a PI animal may be present. This is mainly because there was no work to show that it could be done on an entire population cost effectively. In this program we would outline the steps required to see the kind of returns our research shows. These steps would include the following management steps: individual identification, sampling and testing with a suitable time-effective test (such as ACE) for PI animals, removal of PI animals as promptly as possible. Typically, ear notch samples were taken within three days of arrival in the feedlot, preferably within 1-2 days of arrival. Screening test results were routinely available about 12 to about 36 hours after sampling. Typically, PI animals were removed within one day after the screening results were available, and about 36-72 hours after arrival at the cattle operation.

In certain preferred embodiments, other common health practices such as vaccination programs, deworming and administering growth implants are also performed at arrival in the cattle operation.

This program could alternatively be administered at the farm of origin, dairy farm, auction facility, gathering point, buyer facility, stocker or backgrounding operation. This study showed about $20/head improvement (estimated range of $17-$24) in non-PI animals if PI animals are removed, which is a substantial return at the feedlot level. It would be expected that there would be at least as great a return if the method of the present invention were applied at an earlier stage in the livestock production chain.

The claims should not be read as limited to the described order or elements unless stated to that effect. Therefore, all embodiments that come within the scope and spirit of the following claims and equivalents thereto are claimed as the invention.

1. A method of prophylactic screening of a population of livestock containing subclinical persistently infected animals comprising the steps of:
receiving samples obtained from identified individual animals to be screened;
providing aliquots of the samples;
combining aliquots of the samples to form pooled samples;
testing the pooled samples for presence of a pathogen;
identifying the pooled samples that are pathogen positive, thereby identifying a subset of putative positive samples;
testing aliquots from the putative positive samples to produce a set of pathogen positive samples,
identifying the individual animals that provided the pathogen positive samples, thereby identifying subclinical pathogen positive individual animals within the population of livestock, and reporting the test results about twelve to about twenty-four hours after the samples after receiving the samples.

2. The method of claim 1 wherein the test results are reported results about twelve hours after receiving the samples to be screened.

3. The method of claim 1 wherein the information regarding the identity of the individual pathogen positive animals is used in a method of managing livestock in a cattle operation.

4. The method of claim 3 wherein the cattle operation is a seedstock operation, a cow-calf operation, a stocker operation, a backgrounding operation, a feedlot operation or a dairy operation.

5. The method of claim 3 wherein the identified pathogen positive animals are removed from the population of livestock.

6. The method of claim 1 wherein the total number of tests performed is less that 30% of the number of samples to be tested.

7. The method of claim 1, wherein y tests are performed on x samples, where y is z₁+z₂, where z₁ is the maximum number of tests of pooled samples and is the integer that is greater or equal to x/b, where x is the number of samples and b is the number of aliquots from individual samples that are combined to make a pooled sample, and z₂ is the maximum number of tests of individual samples and is the integer that is greater or equal to abx, where a is the estimated prevalence of positive animals in the screened population, x is the number of samples and b is the number of aliquots from individual samples that are combined to make a pooled sample.

8. The method of claim 1 wherein the sampling and prophylactic screening of a population of livestock is completed in less than 48 hours.

9. The method of claim 1 wherein the testing of the pooled samples is performed using an antigen capture ELISA test.

10. The method of claim 1 wherein the testing of aliquots from the putative positive samples is performed using an antigen capture ELISA test.

11. A method of improving the performance and health of cattle in a cattle operation, comprising the steps of:
keeping the cattle separated by source in arrival groups;
performing prophylactic screening of all cattle for the presence of bovine viral diarrhea virus;
removing bovine viral diarrhea virus positive animals from the arrival groups within about 1.5 to about 3 days after arrival in the cattle operation; and
feeding and managing the bovine viral diarrhea virus negative animals.

12. The method of claim 11 further comprising the step of:
feeding and managing the removed bovine viral diarrhea virus positive animals.

13. The method of claim 11 further comprising the step of:
performing intake processing, wherein intake processing includes at least one of the steps of identifying individual animals, weighing identified individual animals and performing ear notch sampling.

14. (canceled)

15. (canceled)

16. The method of claim 11 wherein the cattle operation is a feedlot, a farm of origin, a dairy farm, an auction facility, a gathering point, a buyer facility, a stocker or a backgrounding operation.

17. The method of claim 13 wherein the step of performing prophylactic screening for bovine viral diarrhea virus infection comprises the steps of:
receiving at least one ear notch sample obtained from an identified individual animal;
preparing the ear notch sample;
aliquoting the prepared sample;
pooling aliquots from b prepared samples to form at least one pooled sample, where b is an integer from 2 to 10, inclusive;
testing the pooled sample for the presence of bovine viral diarrhea virus;
determining if the tested pooled sample is positive for bovine viral diarrhea virus infection;
obtaining an individual aliquot from each prepared sample that contributed to a positive pooled sample;
testing the individual aliquot for bovine viral diarrhea virus infection;
determining if a pooled sample is positive for bovine viral diarrhea virus infection;
correlating the prepared sample with the individually identified animal; and reporting the test results about twelve to about twenty-four hours after receiving the samples.

18. The method of claim 17 wherein the test results are reported about 12 hours after receiving the samples.

19. The method of claim 17 wherein the step of preparing the ear notch samples includes the step of contacting the ear notch samples with phosphate buffered saline to form a prepared sample comprising an ear notch sample and phosphate buffered saline.
